(19) United States
(12) Patent Application Publication
(10) Pub. No.: US 2023/0177634 A1
(43) Pub. Date: Jun. 8, 2023

(54) PREDICTING AND EXPLAINING THE EFFECTIVENESS OF SOCIAL PROGRAMS

(71) Applicant: International Business Machines Corporation, Armonk, NY (US)

(72) Inventors: Natalia Mulligan, Dublin (IE); Marco Luca Sbodio, Castaheany (IE); Joao H. Bettencourt-Silva, Dublin (IE); Gabriele Picco, Dublin (IE); Vanessa Lopez Garcia, Dublin (IE); Conor Patrick Cullen, Clontarf (IE)

(21) Appl. No.: 17/643,197

(22) Filed: Dec. 8, 2021

(51) Int. Cl. G06Q 50/22; G16H 10/60

(52) U.S. Cl. CPC G06Q 50/22; G16H 10/60

(57) ABSTRACT

In an approach for predicting an effectiveness of a given social program for a given patient at one or more points in time and for providing a score indicating the accuracy of the prediction and an explanation of the prediction, a processor receives a request from a user. Responsive to determining a social program database contains historical data on the given social program, a processor analyzes a set of patients associated with the given social program. A processor predicts the effectiveness of the given social program for the given patient at the one or more points in time using a prediction model trained to predict an effectiveness score and a confidence score. A processor outputs a prediction of the effectiveness of the given social program for the given patient at the one or more points in time to the user.

### BACKGROUND OF THE INVENTION

The present invention relates generally to the field of data processing, and more particularly to predicting and explaining the effectiveness of social programs.

Social programs are governmental programs designed to protect citizens from the economic risks and insecurities of life. Social programs ensure that a person’s basic needs (i.e., food, shelter, education, and healthcare) are met by providing the person with benefits. The most common types of benefits provided by social programs include, but are not limited to, education and childcare assistance, cash assistance, food assistance, housing subsidies, energy and utilities subsidies, and health insurance. More particularly, in the United States, the most prominent social programs provide benefits such as primary and secondary public education, subsidies for higher education, unemployment and disability insurance, subsidies for eligible low-wage workers, Social Security, pensions, Supplemental Nutrition Assistance Program benefits, subsidies for housing, health insurance programs, Medicare, Medicaid, and the Children’s Health Insurance Program. Social programs vary in eligibility with some, such as primary and secondary public education, available to all, while others, such as housing subsidies, available only to a subsegment of the population (i.e., to the elderly or retirees, the sick or invalid, dependent survivors, mothers, the unemployed, the work-injured, or families).

Social programs are not without their problems. Assigning benefits to those people who truly need the assistance and receiving assurance that the benefits provided to those people will be life changing is a problem. Current capabilities are limited to performing triages to determine a person’s eligibility for and entitlement to a social program as well as determining the productivity of the social program by applying the rules of an engine to real-world data (e.g., by applying rules of an engine based on household composition or income levels).

For example, in the patent application entitled Health Care Eligibility Verification and Settlement Systems and Methods (U.S. Pat. Appl. Pub. No. US2014/0316798A1), a method utilizing a point-of-care terminal to process “a healthcare transaction by a healthcare provider” is described. The method “includes a reader configured to read information from a healthcare eligibility and settlement presentation instrument associated with a patient and a processor configured to process a healthcare transaction based on the information, wherein the healthcare transaction includes at least one of a healthcare eligibility verification transaction and a healthcare settlement transaction.”

In another example, in the patent application entitled Points-Based Reward Program for Improving Medication Adherence and Outcomes (U.S. Pat. Appl. Pub. No. US2014/0052476A1), a method “for improving medication adherence and outcomes” is described. The method “includes registering a patient in a predetermined manner with a web site thereby creating an associated user profile, manually entering patient medication data and patient health data associated with the user profile, automatically acquiring from a third party and then storing patient medication data and patient health data associated with the user profile and assigning a points based reward.”

In yet example, in the patent application entitled Methods for Predicting an Individual’s Clinical Treatment Outcome from Sampling a Group of Patient’s Biological Profiles (U.S. Pat. Appl. Pub. No. US2016/0306917A1), a method that predicts “an individual’s treatment outcome from a sampling of a group of patients’ biological profiles” is described. The method involves “obtaining the results of a correlation between patient biological profile information and the predictive information of post-treatment efficacy for a particular drug treatment selected from one or more treatments for the cancer.”

Despite these works, the current capabilities fail to address the need for a system and method capable of providing evidence on the effectiveness of the benefits of a social program for a given patient, family, or cohort at a given time horizon.

### SUMMARY

Aspects of an embodiment of the present invention disclose a method, computer program product, and computer system for predicting an effectiveness of a given social program for a given patient at one or more points in time and for providing a score indicating the accuracy of the prediction and an explanation of the prediction. A processor receives a request to predict an effectiveness of a given social program for a given patient at one or more points in time from a user. Responsive to determining a social program database contains historical data on the given social program, a processor analyzes a set of patients associated with the given social program. A processor predicts the effectiveness of the given social program for the given patient at the one or more points in time using a prediction model trained to predict an effectiveness score and a confidence score. A processor outputs a prediction of the effectiveness of the given social program for the given patient at the one or more points in time to the user.

In some aspects of an embodiment of the present invention, a processor requests feedback on the prediction of the effectiveness of the given social program for the given patient at the one or more points in time. A processor receives the feedback on the prediction of the effectiveness of the given social program for the given patient at the one or more points in time. A processor integrates the feedback on the prediction of the effectiveness of the given social program for the given patient at the one or more points in time to refine the prediction model.

In some aspects of an embodiment of the present invention, patient data includes socio-demographic information of the given patient, an Electronic Health Record of the given patient, a record of past and present social program the given patient participated in, and one or more Activities of Daily Living of the given patient.

In some aspects of an embodiment of the present invention, the historical data on the given social program includes an enrollment history of the given social program and one or more previous outcomes of the given social program.

In some aspects of an embodiment of the present invention, a processor calculates a first measure of similarity between a profile of the given patient and each profile of the set of patients associated with the given social program. A processor selects a subset of patients from the set of patients associated with the given social program who exceed a pre-set threshold of similarity.

In some aspects of an embodiment of the present invention, the prediction of the effectiveness of the given social program for the given patient at the one or more points in time is comprised of the effectiveness score, the confidence score, and an explanation of the prediction of the effectiveness of the given social program for the given patient at the one or more points in time, wherein the explanation of the prediction of the effectiveness of the given social program for the given patient at the one or more points in time includes one or more factors used to calculate the effectiveness score.

In some aspects of an embodiment of the present invention, a processor defines a first indicator function for each patient of the subset of patients selected at the one or more points in time using a default function. A processor aggregates a plurality of first indicator functions defined for each patient of the subset of patients selected at the one or more points in time. A processor calculates the effectiveness score using a value of the plurality of first indicator functions aggregated. A processor calculates the confidence score using the value of the plurality of first indicator functions aggregated. A processor compiles an explanation of one or more factors that contributed to a calculation of the effectiveness score and a calculation of the confidence score using domain knowledge. A processor compiles the explanation of the one or more factors that contributed to the calculation of the effectiveness score and the calculation of the confidence score using a machine learning explanation and interpretation technique.

In some aspects of an embodiment of the present invention, the default function is a historical outcome of the given social program for the subset of patients from the set of patients associated with the given social program.

In some aspects of an embodiment of the present invention, responsive to determining the social program database does not contain historical data on the given social program, a processor identifies a set of social programs similar to the given social program. A processor calculates a second measure of similarity between a feature of the given social program and a feature of each social program in the set of social programs similar to the given social program. A processor selects a subset of social programs from the set of social programs similar to the given social program that exceed the pre-set threshold of similarity. A processor analyzes the set of patients associated with the subset of social programs selected. A processor calculates a third measure of similarity between the profile of the given patient and each profile of the set of patients associated with the subset of social programs selected. A processor selects a subset of patients from the set of patients associated with the subset of social programs selected who exceed the pre-set threshold of similarity. A processor predicts the effectiveness of the given social program for the given patient at the one or more points in time using the prediction model trained to predict the effectiveness score and the confidence score. A processor outputs the prediction of the effectiveness of the given social program for the given patient at the one or more points in time to the user.

In some aspects of an embodiment of the present invention, a processor defines a second indicator function for the subset of patients selected and the subset of social programs selected at the one or more points in time using the default function. A processor aggregates a plurality of second indicator functions defined for the subset of patients selected and the subset of social programs selected at the one or more points in time. A processor calculates the effectiveness score using a value of the plurality of second indicator functions aggregated. A processor calculates the confidence score using the value of the plurality of second indicator functions aggregated. A processor compiles the explanation of the one or more factors that contributed to the calculation of the effectiveness score and the calculation of the confidence score at the one or more points in time using domain knowledge. A processor compiles the explanation of the one or more factors that contributed to the calculation of the effectiveness score and the calculation of the confidence score at the one or more points in time using the machine learning explanation and interpretation technique.

These and other features and advantages of the present invention will be described in, or will become apparent to those of ordinary skill in the art in view of, the following detailed description of the example embodiments of the present invention.

### BRIEF DESCRIPTION OF THE DRAWINGS

FIG. 1 is a block diagram illustrating a distributed data processing environment, in accordance with an embodiment of the present invention;

FIG. 2 is a flowchart illustrating the operational steps for a setup component of an effectiveness prediction and explanation program, on a server within the distributed data processing environment of FIG. 1, in accordance with an embodiment of the present invention;

FIG. 3 is a flowchart illustrating the operational steps of the effectiveness prediction and explanation program, on the server within the distributed data processing environment of FIG. 1, in accordance with an embodiment of the present invention; and

FIG. 4 is a block diagram illustrating the components of the server within the distributed data processing environment of FIG. 1, in accordance with an embodiment of the present invention.

### DETAILED DESCRIPTION

Embodiments of the present invention recognize that social programs are governmental programs designed to protect citizens from the economic risks and insecurities of life. Embodiments of the present invention recognize that social programs ensure that a person’s basic needs (i.e., food, shelter, education, and healthcare) are met by providing the person with benefits. Embodiments of the present invention recognize that the benefits provided by social programs should be a springboard of upward social mobility. That means, the benefits provided by social programs should give the person the type of assistance that will be life changing and will allow the person to make substantial long-term investments, rather than simply provide for the person’s short-term needs.

Embodiments of the present invention recognize that current capabilities are limited to performing triages to determine a person’s eligibility for and entitlement to a social program as well as the productivity of the social program by applying the rules of a model to real-world data (e.g., household composition or income levels). Embodiments of the present invention recognize that current capabilities fail to provide evidence on the effectiveness of the benefits of the social program for a given patient, family, or cohort at a given time horizon.

For example, in the case-control study “Social Determinants of Health and Diabetes: A Scientific Review,” a randomized social experiment was conducted by Felicia Hill-Briggs, Nancy E. Alder, Seth A. Berkowitz, Marshall H. Chin, Tiffany L. Gary-Webb, Ana Navas-Acien, Pamela L. Thornton, Debra Haire-Joshu. In this randomized social experiment, the researchers looked at whether the effectiveness of housing intervention affected a person’s diabetes. More specifically, the researchers used a single mother with one or more children living in public housing within high-poverty census tracts as the target household unit and a move to a low-poverty census tract using an unrestricted voucher as the housing intervention. The researchers found a 21.6% relative reduction in the prevalence of an elevated blood sugar in the target household unit when moved to a low-poverty census tract using traditional unrestricted vouchers.

Therefore, embodiments of the present invention recognize the need for a program that goes beyond the current capabilities. Embodiments of the present invention recognize the need for a social program effectiveness system that comprehends the underlying needs of the populations that receive benefits from social programs with the ultimate goal of reducing a person’s need for benefits, saving costs, and improving outcomes.

Embodiments of the present invention provide a system and method capable of predicting the effectiveness of a given social program for a given patient, family, or cohort at one or more points in time and of providing a score indicating the accuracy of the prediction and an explanation of the prediction.

Implementation of embodiments of the present invention may take a variety of forms, and exemplary implementation details are discussed subsequently with reference to the Figures.

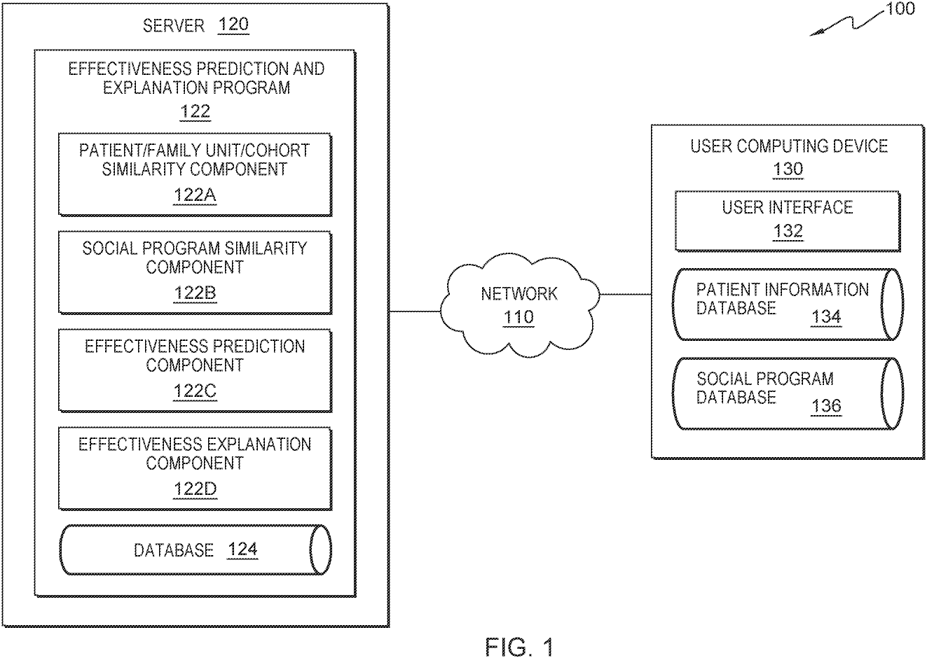

FIG. 1 is a block diagram illustrating a distributed data processing environment, generally designated 100, in accordance with an embodiment of the present invention. In the depicted embodiment, distributed data processing environment 100 includes server 120 and user computing device 130, interconnected over network 110. Distributed data processing environment 100 may include additional servers, computers, computing devices, IoT sensors, and other devices not shown. The term “distributed” as used herein describes a computer system that includes multiple, physically distinct devices that operate together as a single computer system. FIG. 1 provides only an illustration of one embodiment of the present invention and does not imply any limitations with regards to the environments in which different embodiments may be implemented. Many modifications to the depicted environment may be made by those skilled in the art without departing from the scope of the invention as recited by the claims.

Network 110 operates as a computing network that can be, for example, a telecommunications network, a local area network (LAN), a wide area network (WAN), such as the Internet, or a combination of the three, and can include wired, wireless, or fiber optic connections. Network 110 can include one or more wired and/or wireless networks capable of receiving and transmitting data, voice, and/or video signals, including multimedia signals that include data, voice, and video information. In general, network 110 can be any combination of connections and protocols that will support communications between server 120, user computing device 130, and other computing devices (not shown) within distributed data processing environment 100.

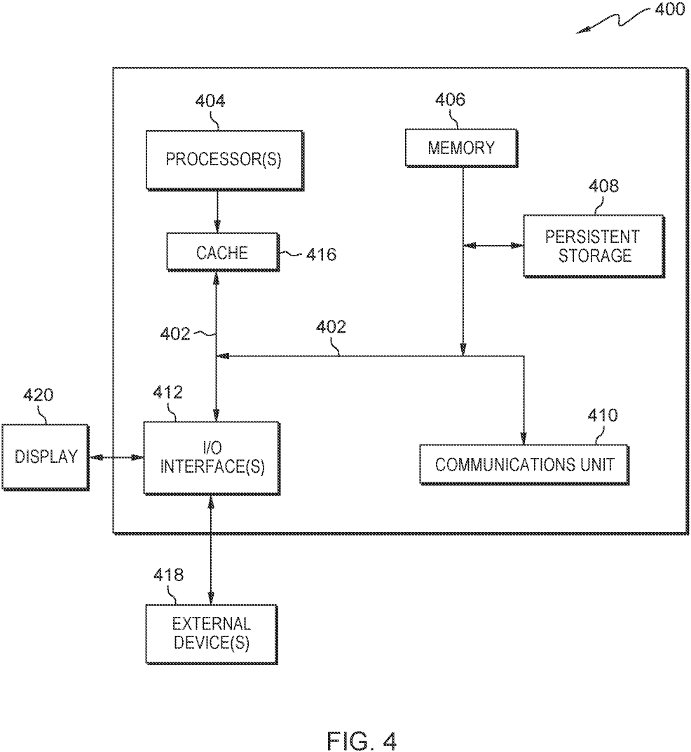

Server 120 operates to run effectiveness prediction and explanation program 122 and to send and/or store data in database 124. In an embodiment, server 120 can send data from database 124 to user computing device 130. In an embodiment, server 120 can receive data in database 124 from user computing device 130. In one or more embodiments, server 120 can be a standalone computing device, a management server, a web server, a mobile computing device, or any other electronic device or computing system capable of receiving, sending, and processing data and capable of communicating with user computing device 130 via network 110. In one or more embodiments, server 120 can be a computing system utilizing clustered computers and components (e.g., database server computers, application server computers, etc.) that act as a single pool of seamless resources when accessed within distributed data processing environment 100, such as in a cloud computing environment. In one or more embodiments, server 120 can be a laptop computer, a tablet computer, a netbook computer, a personal computer, a desktop computer, a personal digital assistant, a smart phone, or any programmable electronic device capable of communicating with user computing device 130 and other computing devices (not shown) within distributed data processing environment 100 via network 110. Server 120 contains effectiveness prediction and explanation program 122, the components of effectiveness prediction and explanation program 122 (i.e., patient similarity component 122A, social program similarity component 122B, effectiveness prediction component 122C, and effectiveness explanation component 122D), and database 124. Server 120 may include internal and external hardware components, as depicted and described in further detail in FIG. 4.

Effectiveness prediction and explanation program 122 operates to predict the effectiveness of a given social program for a given patient, family, or cohort at one or more points in time and to provide a score indicating the accuracy of the prediction and an explanation of the prediction. In the depicted embodiment, effectiveness prediction and explanation program 122 is comprised of patient similarity component 122A, social program similarity component 122B, effectiveness prediction component 122C, and effectiveness explanation component 122D. In the depicted embodiment, effectiveness prediction and explanation program 122 is a standalone program. In another embodiment, effectiveness prediction and explanation program 122 may be integrated into another software product, such as a case management program (i.e., a program that enables a user to streamline data collection, tracking, and reporting by consolidating the data in a central repository while also enabling the user to focus on client management and social service outcomes). In the depicted embodiment, effectiveness prediction and explanation program 122 resides on server 120. In another embodiment, effectiveness prediction and explanation program 122 may reside on user computing device 130 or on another computing device (not shown), provided that effectiveness prediction and explanation program 122 has access to network 110.

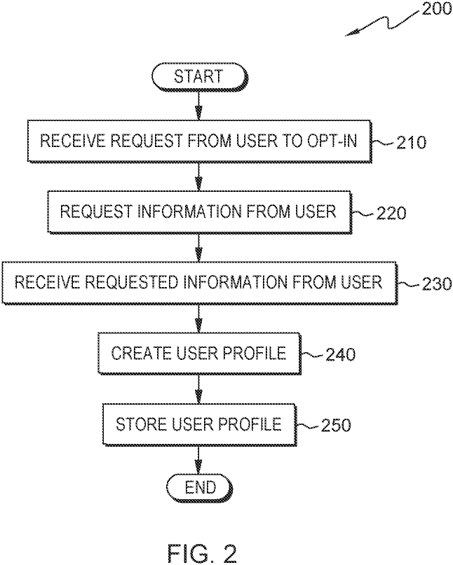

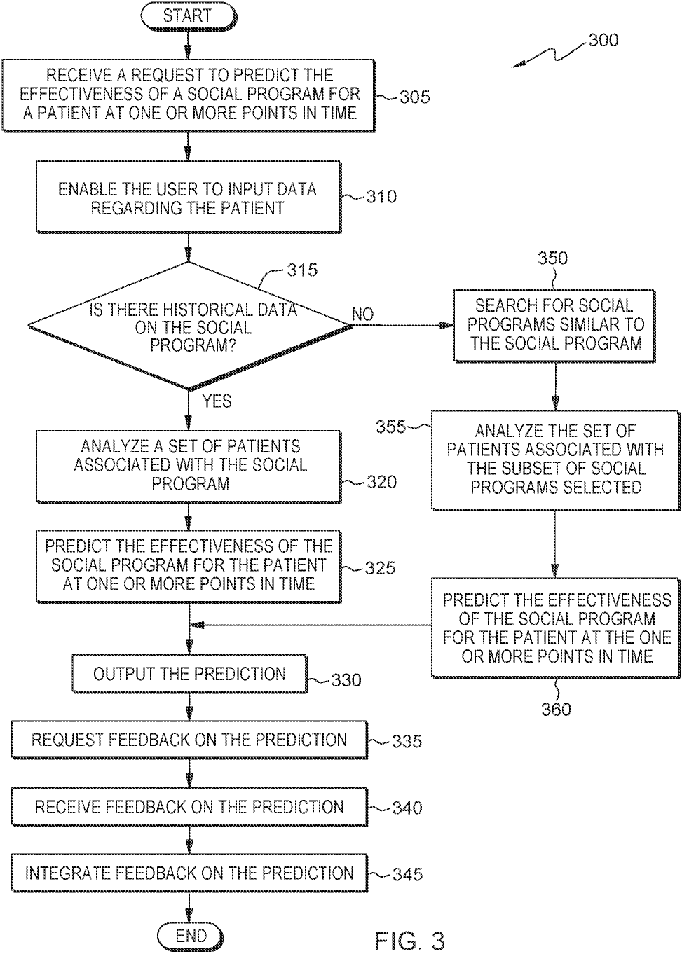

In an embodiment, the user of user computing device 130 registers with server 120. For example, the user completes a registration process (e.g., user validation), provides information to create a user profile, and authorizes the collection, analysis, and distribution (i.e., opts-in) of relevant data on identified computing devices (e.g., on user computing device 130) by server 120 (e.g., via effectiveness prediction and explanation program 122). Relevant data may include, but is not limited to, personal information or data provided by the user or inadvertently provided by the user’s device without the user’s knowledge; and tagged and/or recorded location information of the user (e.g., to infer context (i.e., time, place, and usage) of a location or existence). In an embodiment, the user opts-in or opts-out of certain categories of data collection. For example, the user can opt-in to provide all requested information, a subset of requested information, or no information. In one example scenario, the user opts-in to provide time-based information, but opts-out of providing location-based information (on all or a subset of computing devices associated with the user). In an embodiment, the user opts-in or opts-out of certain categories of data analysis. In an embodiment, the user opts-in or opts-out of certain categories of data distribution. Such preferences can be stored in a database, e.g., database 124. The setup component of effectiveness prediction and explanation program 122 is depicted and described in further detail with respect to FIG. 2. The overall operational steps of effectiveness prediction and explanation program 122 are depicted and described in further detail with respect to FIG. 3.

Database 124 operates as a repository for data received, used, and/or generated by effectiveness prediction and explanation program 122. A database is an organized collection of data. Data includes, but is not limited to, information about user preferences (e.g., general user system settings such as alert notifications for user computing device 130); information about alert notification preferences; the prediction made; and any other data received, used, and/or generated by effectiveness prediction and explanation program 122.

Database 124 can be implemented with any type of device capable of storing data and configuration files that can be accessed and utilized by server 120, such as a hard disk drive, a database server, or a flash memory. In an embodiment, database 124 is accessed by effectiveness prediction and explanation program 122 to store and/or to access the data. In the depicted embodiment, database 124 resides on server 120. In another embodiment, database 124 may reside on another computing device, server, cloud server, or spread across multiple devices elsewhere (not shown) within distributed data processing environment 100, provided that effectiveness prediction and explanation program 122 has access to database 124.

The present invention may contain various accessible data sources, such as database 124, that may include personal and/or confidential data, content, or information the user wishes not to be processed. Processing refers to any operation, automated or unautomated, or set of operations such as collecting, recording, organizing, structuring, storing, adapting, altering, retrieving, consulting, using, disclosing by transmission, dissemination, or otherwise making available, combining, restricting, erasing, or destroying personal and/or confidential company data. Effectiveness prediction and explanation program 122 enables the authorized and secure processing of personal data.

Effectiveness prediction and explanation program 122 provides informed consent, with notice of the collection of personal and/or confidential data, allowing the user to opt-in or opt-out of processing personal and/or confidential data (i.e., personal and/or confidential data that effectiveness prediction and explanation program 122 has currently and/or receives in the future). Consent can take several forms. Opt-in consent can impose on the user to take an affirmative action before personal and/or confidential data is processed. Alternatively, opt-out consent can impose on the user to take an affirmative action to prevent the processing of personal and/or confidential data before personal and/or confidential data is processed. Effectiveness prediction and explanation program 122 provides information regarding personal and/or confidential data and the nature (e.g., type, scope, purpose, duration, etc.) of the processing. Effectiveness prediction and explanation program 122 provides the user with copies of stored personal and/or confidential data. Effectiveness prediction and explanation program 122 allows the correction or completion of incorrect or incomplete personal and/or confidential data. Effectiveness prediction and explanation program 122 allows for the immediate deletion of personal and/or confidential data.

User computing device 130 operates to run user interface 132 through which a user can interact with effectiveness prediction and explanation program 122 on server 120. In an embodiment, user computing device 130 is a device that performs programmable instructions. For example, user computing device 130 may be an electronic device, such as a laptop computer, a tablet computer, a netbook computer, a personal computer, a desktop computer, a smart phone, or any programmable electronic device capable of running user interface 132 and of communicating (i.e., sending and receiving data) with effectiveness prediction and explanation program 122 via network 110. In general, user computing device 130 represents any programmable electronic device or a combination of programmable electronic devices capable of executing machine readable program instructions and communicating with other computing devices (not shown) within distributed data processing environment 100 via network 110. In the depicted embodiment, user computing device 130 contains patient information database 134 and social program database 136.

User interface 132 operates as a local user interface between effectiveness prediction and explanation program 122 on server 120 and a user of user computing device 130. In some embodiments, user interface 132 is a graphical user interface (GUI), a web user interface (WUI), and/or a voice user interface (VUI) that can display (i.e., visually) or present (i.e., audibly) text, documents, web browser windows, user options, application interfaces, and instructions for operations sent from effectiveness prediction and explanation program 122 to a user via network 110. User interface 132 can also display or present alerts including information (such as graphics, text, and/or sound) sent from effectiveness prediction and explanation program 122 to a user via network 110. In an embodiment, user interface 132 is capable of sending and receiving data (i.e., to and from effectiveness prediction and explanation program 122 via network 110, respectively). Through user interface 132, a user can opt-in to effectiveness prediction and explanation program 122; create a user profile; input information; set user preferences and alert notification preferences; input a request; and receive a prediction.

A user preference is a setting that can be customized for a particular user. A set of default user preferences are assigned to each user of effectiveness prediction and explanation program 122. A user preference editor can be used to update values to change the default user preferences. User preferences that can be customized include, but are not limited to, general user system settings, specific user profile settings, alert notification settings, and machine-learned data collection/storage settings.

Specific user profile settings provide a tailored delivery of contextual content. Effectiveness prediction and explanation program 122 delivers the amount of contextual content required by the user, based on the personal preferences set by the user. The amount of contextual content required by the user varies, but may be an unabridged content comparison (i.e., the complete text that has not been cut down or shortened in any form or manner), an abridged content comparison (i.e., a shortened piece of content that does not lose sense of the overall, complete content), a summary content comparison (i.e., a paragraph style summary that conveys all of the points in summary format), or an executive summary of content comparison (i.e., a shortened summary that is about three to five sentences in length).

Machine-learned data is a user’s personalized corpus of data. Machine-learned data includes, but is not limited to, data regarding information and/or products that the user has seen or experienced, past results of iterations of effectiveness prediction and explanation program 122 and a user’s previous response to an alert notification sent by effectiveness prediction and explanation program 122. Effectiveness prediction and explanation program 122 self-learns by tracking user activity and improves with each iteration of effectiveness prediction and explanation program 122. By retaining such data, effectiveness prediction and explanation program 122 ensures that repetitive information is not generated and sent to the user. Instead, effectiveness prediction and explanation program 122 bypasses the repetitive information and locates new information for the user.

Patient information database 134 operates as a repository for patient information data received, used, and/or generated by the user. Data stored in patient information database 134 may include, but is not limited to, socio-demographic information of a patient, family unit, and cohort; Electronic Health Records (EHR) of a patient, family unit, and cohort; past and present social program history of a patient, family unit, and cohort (i.e., social programs that a patient, family unit, and cohort has been enrolled in or is currently enrolled in); and Activities of Daily Living (ADL) of a patient, family unit, and cohort. In the depicted embodiment, patient information database 134 resides on user computing device 130. In another embodiment, patient information database 134 may reside remotely from user computing device 130 such as in another location including, but not limited to, cloud storage or another server.

Social program database 136 operates as a repository for social program data received, used, and/or generated by the user. Data stored in social program database 136 includes, but is not limited to, a description of a social program; historical data of a social program (i.e., enrollment history and outcomes of social program), criteria for eligibility for a social program; and other documents regarding a social program (i.e., a list of activities, goals, objectives, benefits, and rules). In the depicted embodiment, social program database 136 resides on user computing device 130. In another embodiment, social program database 136 may reside remotely from user computing device such as in another location including, but not limited to, cloud storage or another server.

FIG. 2 is a flowchart, generally designated 200, illustrating the operational steps for a setup component of effectiveness prediction and explanation program 122 (hereinafter referred to as “program 122”) on server 120 within distributed data processing environment 100 of FIG. 1, in accordance with an embodiment of the present invention. In an embodiment, program 122 completes a one-time setup with a user. The one-time setup allows for program 122 to capture relevant information about the user to create a user profile. In an embodiment, program 122 receives a request from the user to opt-in. In an embodiment, program 122 requests information from the user. In an embodiment, program 122 receives the requested information from the user. In an embodiment, program 122 creates a user profile. In an embodiment, program 122 stores the user profile. It should be appreciated that the process depicted in FIG. 2 illustrates one possible iteration of program 122, which may be repeated for each opt-in request received by program 122.

In step 210, program 122 receives a request from a user to opt-in. A user may include, but is not limited to, a local, regional, or state level government agency or a health and social welfare institution or a representative of those agencies or institutions. In an embodiment, program 122 receives a request from a user to opt-in to program 122. In an embodiment, program 122 receives a request from a user to opt-in to program 122 through user interface 132 of user computing device 130. By opting-in, the user agrees to share at least some data with database 124.

In step 220, program 122 requests information from the user. In an embodiment, responsive to receiving a request from a user to opt-in, program 122 requests information from the user. Information requested from the user may include, but is not limited to, information about user preferences; information about alert notification preferences; patient information data received, used, and/or generated by the user; and social program data received, used, and/or generated by the user. In an embodiment, program 122 requests information from the user to create a user profile. In an embodiment, program 122 requests information from the user through user interface 132 of user computing device 130.

In step 230, program 122 receives the requested information from the user. In an embodiment, responsive to requesting information from the user, program 122 receives the requested information from the user. In an embodiment, program 122 receives the requested information from the user through user interface 132 of user computing device 130.

In step 240, program 122 creates a user profile. In an embodiment, responsive to receiving the requested information from the user, program 122 creates a user profile. In an embodiment, program 122 creates a user profile for the user. In an embodiment, program 122 creates a user profile with information input by the user during setup regarding the user (i.e., information necessary to create a user profile), as well as user preferences and alert notification preferences.

In step 250, program 122 stores the user profile. In an embodiment, responsive to creating a user profile, program 122 stores the user profile. In an embodiment, program 122 stores the user profile in a database, e.g., database 124.

FIG. 3 is a flowchart, generally designated 300, illustrating the operational steps of program 122 on server 120 within distributed data processing environment 100 of FIG. 1, in accordance with an embodiment of the present invention. In an embodiment, program 122 operates to predict the effectiveness of a given social program for a given patient, a given family, or a given cohort at one or more points in time and to provide a score indicating the accuracy of the prediction and an explanation of the prediction. It should be appreciated that the process depicted in FIG. 3 illustrates one possible iteration of the process flow, which may be repeated for each request received by program 122.

In step 305, program 122 receives a request. In an embodiment, program 122 receives a request to predict the effectiveness of a given social program (also referred to as “S”) for a given patient (also referred to as “P”) at one or more points in time (e.g., a short term, a medium term, and a long term, e.g., 7 days, 6 months, 1 year). In another embodiment, program 122 receives a request to predict the effectiveness of a given social program for a given family unit (also referred to as “U”) at one or more points in time. In another embodiment, program 122 receives a request to predict the effectiveness of a given social program for a given cohort (also referred to as “C”) (i.e., a group of people, e.g., associates or companions) at one or more points in time. The given social program may be a social care or health care intervention or action or a combination of social care and health care interventions and actions for which the given patient, the given family unit, or the given cohort may be eligible or for which the given patient, the given family unit, or the given cohort may not be eligible but is still a relevant social care or health care intervention or action. In an embodiment, program 122 receives a request manually inputted by a user into user interface 132 of user computing device 130.

For example, program 122 receives a request to predict the effectiveness of a given social program, a combination of social care and health care interventions and actions, for a given family unit in the short term and in the long term.

In step 310, program 122 enables the user to input data regarding the given patient. In an embodiment, responsive to receiving a request, program 122 enables the user to input data regarding the given patient (hereinafter referred to as “patient data”). Patient data includes, but is not limited to, socio-demographic information of the given patient, EHRs of the given patient, past and present social program history of the given patient (i.e., social programs that the given patient has been enrolled in or is currently enrolled in), and ADLs of the given patient. In an embodiment, program 122 enables the user to input patient data from patient information database 134 on user computing device 130.

In another embodiment, program 122 enables the user to input data regarding the given family unit (hereinafter referred to as “family unit data”) from patient information database 134 on user computing device 130. Family unit data is data collected by a social care worker and input into patient information database 134 on user computing device 130. Family unit data includes data similar to patient data (i.e., socio-demographic information of the given family unit, EHRs of the given family unit, past and present social program history of the given family unit, and ADLs of the given family unit).

In another embodiment, program 122 enables the user to input data regarding the given cohort (hereinafter referred to as “cohort data”) from patient information database 134 on user computing device 130. Cohort data is data collected by a social care worker and input into patient information database 134 on user computing device 130. Cohort data includes data similar to patient data (i.e., socio-demographic information of the given cohort, EHRs of the given cohort, past and present social program history of the given cohort, and ADLs of the given cohort).

Continuing the example from above, program 122 enables the user to input data regarding the given family unit. From the family unit data, program 122 finds that the given family unit is composed of a single mother and her two children, that the mother is unemployed and therefore the given family unit falls into the low-income household category, that the given family unit faces food insecurity, and that the given family unit does not own a car and therefore faces transportation issues.

In decision 315, program 122 determines whether there is historical data on the given social program. In an embodiment, responsive to enabling the user to input data regarding the given patient, program 122 determines whether there is historical data on the given social program. In an embodiment, program 122 determines whether there is historical data on the given social program stored in social program database 136. Historical data on the given social program includes, but is not limited to, an enrollment history of the given social program and an outcome of the given social program. If program 122 determines there is historical data on the given social program stored in social program database 136 (decision 315, YES branch), then program 122 proceeds to step 320, analyzing a set of patients associated with the given social program. If program 122 determines there is no historical data on the given social program stored in social program database 136 (decision 315, NO branch), then program 122 proceeds to step 350, searching for one or more social programs similar to the given social program.

Additional data on the given social program includes, but is not limited to, a description of the given social program, criteria for eligibility for the given social program, and other documents regarding the given social program (i.e., a list of activities, goals, objectives, benefits, and rules of the given social program).

Continuing the example from above, the given social program is a combination of social care and health care interventions and actions for which the given family unit may be eligible and for which the given family unit is not eligible but the given social program is still a relevant social care or health care intervention and action. Based on the triage of the given family unit, program 122 determines that the given family unit is eligible for a cash assistance program, a community garden program, an employment counselor, and a bus pass to work and to the market. Program 122 also determines social program database 136 contains historical data on the given social program.

In step 320, patient similarity component 122A of program 122 analyzes a set of patients associated with the given social program. In an embodiment, responsive to determining there is historical data on the given social program stored in social program database 136, patient similarity component 122A of program 122 analyzes a set of patients associated with the given social program. In an embodiment, patient similarity component 122A of program 122 calculates a measure of similarity. The measure of similarity may be equal to a distance between a profile of the given patient and each profile of the set of patients associated with the given social program. The measure of similarity is defined as a vector in an embedding space. In an embodiment, patient similarity component 122A of program 122 selects a subset of patients from the set of patients (also referred to as P′) associated with the given social program who exceed a pre-set threshold of similarity.

In step 325, effectiveness prediction component 122C of program 122 predicts the effectiveness of the given social program for the given patient at the one or more points in time. In an embodiment, responsive to analyzing the set of patients associated with the given social program, effectiveness prediction component 122C of program 122 predicts the effectiveness of the given social program for the given patient at the one or more points in time (hereinafter referred to as the “prediction”). The prediction includes, but is not limited to, an effectiveness score, which summarizes how effective the given social program could be for the given patient at the one or more points in time; a confidence score, which indicates the accuracy of the effectiveness score; and an explanation of the prediction. The results of the prediction are represented in a table. The table is comprised of one or more columns (i.e., one column for the effectiveness score, one column for the confidence score, and one column for the explanation of the prediction) and one or more rows (i.e., one row for each point in time).

In an embodiment, effectiveness prediction component 122C of program 122 defines an indicator function for each patient of the subset of patients selected at one or more points in time. The indicator function may be, for example, an outcome of the given social program, a length of time the given social program will support the given patient, or an amount of money the given patient will receive from the given social program. In an embodiment, effectiveness prediction component 122C of program 122 defines the indicator function for each patient of the subset of patients selected at one or more points in time using a default function (i.e., if the indicator function is not received as an input). The default function, for example, is a historical outcome of the given social program for the subset of patients selected at the one or more points in time, which may be represented with a finite set of categorical values. In another embodiment, effectiveness prediction component 122C of program 122 defines the indicator function for each patient of the subset of patients selected at one or more points in time using the indicator function for the subset of patients selected (i.e., as a whole) at one or more points in time received as an input.

In an embodiment, effectiveness prediction component 122C of program 122 aggregates the plurality of indicator functions defined for each patient of the subset of patients selected at one or more points in time. The indicator function defined for the subset of patients selected (i.e., as a whole) at the one or more points in time is defined as (F(P′,S)). In an embodiment, effectiveness prediction component 122C of program 122 inserts the indicator function defined for the subset of patients selected (i.e., as a whole) at the one or more points in time into a table.

In an embodiment, effectiveness prediction component 122C of program 122 calculates the effectiveness score of the given social program for the given patient at the one or more points in time using the value of the plurality of indicator functions aggregated. The effectiveness score of the given social program for the given patient at the one or more points in time is defined as (F(P,S)) and is equal to the value of the plurality of indicator functions aggregated at the one or more points in time. The one or more points in time used to calculate the effectiveness score are the same one or more points in time used to define the indicator function. In an embodiment, effectiveness prediction component 122C of program 122 calculates the effectiveness score of the given social program for the given patient at the one or more points in time using a prediction model trained to calculate the values of the effectiveness scores and the confidence scores (i.e., (F(P,S))) over time. In an embodiment, effectiveness prediction component 122C of program 122 inserts the effectiveness score of the given social program for the given patient at the one or more points in time into the “effectiveness score” column of the table.

In an embodiment, effectiveness prediction component 122C of program 122 calculates the confidence score (i.e., the accuracy of the effectiveness score of the given social program for the given patient at the one or more points in time). The one or more points in time used to calculate the confidence score are the same one or more points in time used to define the indicator function. In an embodiment, effectiveness prediction component 122C of program 122 calculates the confidence score using a prediction model trained to predict (F(P,S)) values over time. In an embodiment, effectiveness prediction component 122C of program 122 inserts the confidence score for the one or more points in time into the “confidence score” column of the table.

In an embodiment, effectiveness explanation component 122D of program 122 compiles an explanation. In an embodiment, effectiveness explanation component 122D of program 122 compiles an explanation of the one or more factors that contributed to the calculation of the effectiveness score and the confidence score. In an embodiment, effectiveness explanation component 122D of program 122 compiles an explanation using domain knowledge. Domain knowledge includes, but is not limited to, a knowledge base (KB), an ontology, a taxonomy, a set of statistics on a social domain, and a set of statistics on a clinical domain. In an embodiment, effectiveness explanation component 122D of program 122 compiles an explanation using a machine learning explanation and interpretation technique.

In an embodiment, effectiveness prediction component 122C of program 122 ranks the one or more social care or health care interventions or actions (i.e., the one or more social or health care programs) if the given social program is a combination of social care and health care interventions and/or actions for which the given patient, the given family unit, or the given cohort may be eligible or for which the given patient, the given family unit, or the given cohort may not be eligible but is still a relevant social care or health care intervention or action.

Continuing the example from above, effectiveness prediction component 122C of program 122 finds that, in the short term, the cash assistance program has an effectiveness score of 0.9 because the cash assistance program will allow the given family unit to buy food and fix the immediate household problem of food insecurity; that a bus pass to work and to the market has an effectiveness score of 0.5 because the given family unit lives in a food desert and, therefore, there is no bus available to transport the given family unit to work and to the market and because a bus pass will not fix the given family unit’s problem of food insecurity; and that an employment counselor has an effectiveness score of 0.3 because an employment counselor will not fix the given family unit’s problem of food insecurity. Effectiveness prediction component 122C of program 122 finds that, in the long term, an employment counselor has an effectiveness score of 0.9 because an employment counsel can assist the mother with finding a job that would lift the given family unit out of the low income category and diminish the given family unit’s dependency on the cash assistance program; that child care has an effectiveness score of 0.7 because having child-care in place will remove a barrier of the mother returning to work; and that the cash assistance program has an effectiveness score of 0.5 because the cash assistance program is not cost effective and receiving the cash assistance program for the long-term will create a dependency.

In step 350, social program similarity component 122B of program 122 searches for a set of social programs similar to the given social program. In an embodiment, responsive to determining there is no historical data on the given social program stored in social program database 136, social program similarity component 122B of program 122 searches for a set social programs similar to the given social program. In an embodiment, social program similarity component 122B of program 122 searches for a set of social programs similar to the given social program in social program database 136.

In an embodiment, social program similarity component 122B of program 122 identifies a set of social programs similar to the given social program. In an embodiment, social program similarity component 122B of program 122 calculates a measure of similarity. The measure of similarity may be equal to a distance between one or more features of the given social program and one or more features of each social program in the set of social programs similar to the given social program. The measure of similarity is defined as a vector in an embedding space. The measure of similarity may also be equal to a distance between vectors representing the social programs in the embedding space. In an embodiment, social program similarity component 122B of program 122 selects a subset of social programs from the set of social programs (also referred to as S′) similar to the given social program that exceed a pre-set threshold of similarity.

In step 355, patient similarity component 122A of program 122 analyzes the set of patients associated with the subset of social programs selected. In an embodiment, responsive to searching for a set of social programs similar to the given social program, patient similarity component 122A of program 122 analyzes the set of patients associated with the subset of social programs selected. In an embodiment, patient similarity component 122A of program 122 calculates a measure of similarity. The measure of similarity may be equal to a distance between the profile of the given patient and each profile of the set of patients associated with the subset of social programs selected. The measure of similarity is defined as a vector in an embedding space. In an embodiment, patient similarity component 122A of program 122 selects a subset of patients from the set of patients associated with the subset of social programs selected who exceed a pre-set threshold of similarity (also referred to as P′).

In step 360, effectiveness prediction component 122C of program 122 predicts the effectiveness of the given social program for the given patient at one or more points in time. In an embodiment, responsive to analyzing the set of patients associated with the subset of social programs selected, effectiveness prediction component 122C of program 122 predicts the effectiveness of the given social program for the given patient at one or more points in time (i.e., the “prediction”). The prediction made in step 360 differs from the prediction made in step 325. The prediction made in step 360 is based on the subset of social programs from the set of social programs similar to the given social program and the set of patients associated with the subset of social programs selected as explained below. The prediction made in step 325 is based on the subset of patients selected from the set of patients associated with the given social program as explained above.

In an embodiment, effectiveness prediction component 122C of program 122 defines the indicator function for the subset of patients selected and the subset of social programs selected at one or more points in time. In an embodiment, effectiveness prediction component 122C of program 122 defines the indicator function for the subset of patients selected and the subset of social programs selected at one or more points in time using a default function (i.e., if the indicator function is not received as an input). In another embodiment, effectiveness prediction component 122C of program 122 defines the indicator function for the subset of patients selected and the subset of social programs selected at one or more points in time using the indicator function for the subset of patients selected and the subset of social programs selected (i.e., as a whole) at one or more points in time received as an input.

In an embodiment, effectiveness prediction component 122C of program 122 aggregates the indicator functions defined for the subset of patients selected in each social program of the subset of social programs selected at one or more points in time. The indicator function defined for the subset of patients selected and the subset of social programs selected (i.e., as a whole) at the one or more points in time is defined as (F(P′,S′)). That means, the value (i.e., the effectiveness score and the confidence score) of the indicator function is equal to the aggregated value of the subset of patients selected in each social program of the subset of social programs selected at one or more points in time.The value of the indicator function may not be the same for different social programs in the subset of social programs selected.

In an embodiment, effectiveness prediction component 122C of program 122 inserts the indicator function defined for the subset of patients selected and the subset of social programs selected (i.e., as a whole) at the one or more points in time into the table.

In an embodiment, effectiveness prediction component 122C of program 122 calculates the effectiveness score of the given social program for the given patient at the one or more points in time using the value of the plurality of indicator functions aggregated. The effectiveness score of the given social program for the given patient at the one or more points in time is defined as (F(P′,S′)) and is equal to the value of the plurality of indicator functions aggregated at the one or more points in time. The one or more points in time used to calculate the effectiveness score are the same one or more points in time used to define the indicator function. In an embodiment, effectiveness prediction component 122C of program 122 calculates the effectiveness score of the given social program for the given patient at the one or more points in time using a prediction model trained to calculate the values of the effectiveness scores and the confidence scores (i.e., (F(P,S))) over time. In an embodiment, effectiveness prediction component 122C of program 122 inserts the effectiveness score of the given social program for the given patient at the one or more points in time into the “effectiveness score” column of the table.

In an embodiment, effectiveness prediction component 122C of program 122 calculates the confidence score (i.e., the accuracy of the effectiveness score of the given social program for the given patient at the one or more points in time). The one or more points in time used to calculate the confidence score are the same one or more points in time used to define the indicator function. In an embodiment, effectiveness prediction component 122C of program 122 calculates the confidence score using a prediction model trained to predict (F(P,S)) values over time. In an embodiment, effectiveness prediction component 122C of program 122 inserts the confidence score for the one or more points in time into the “confidence score” column of the table.

In an embodiment, effectiveness explanation component 122D of program 122 compiles an explanation. In an embodiment, effectiveness explanation component 122D of program 122 compiles an explanation of the one or more factors that contributed to the calculation of the effectiveness score and the confidence score. In an embodiment, effectiveness explanation component 122D of program 122 compiles an explanation using domain knowledge. In an embodiment, effectiveness explanation component 122D of program 122 compiles an explanation using a machine learning explanation and interpretation technique.

Returning to step 330, program 122 outputs the prediction. In an embodiment, responsive to predicting the effectiveness of the given social program for the given patient at one or more points in time (i.e., step 325 and step 360), program 122 outputs the prediction. In an embodiment, program 122 outputs the prediction as an alert notification. In an embodiment, program 122 outputs the prediction to the user through user interface 132 of user computing device 130. In an embodiment, program 122 stores the prediction in a database (e.g., database 124).

In step 335, program 122 requests feedback on the prediction. In an embodiment, responsive to outputting the prediction, program 122 requests feedback on the prediction. In an embodiment, program 122 requested feedback on the prediction from the user through user interface 132 of user computing device 130.

In step 340, program 122 receives feedback on the prediction. In an embodiment, responsive to requesting feedback on the prediction, program 122 receives feedback on the prediction. In an embodiment, program 122 receives feedback on the prediction from the user through user interface 132 of user computing device 130. The feedback on the prediction includes, but is not limited to, a quantitative representation of both the usefulness and the accuracy of the prediction and an explanation of the prediction.

In step 345, program 122 integrates the feedback on the prediction. In an embodiment, responsive to receiving feedback on the prediction, program 122 integrates the feedback on the prediction. In an embodiment, program 122 refines the prediction model trained to predict (F(P,S)) values over time with the feedback on the prediction received. In an embodiment, program 122 refines the prediction model trained to predict (F(P,S)) values over time with the feedback on the prediction received in order to improve the accuracy of future predictions.

FIG. 4 is a block diagram illustrating the components of computing device 400 within distributed data processing environment 100 of FIG. 1, in accordance with an embodiment of the present invention. It should be appreciated that FIG. 4 provides only an illustration of one implementation and does not imply any limitations with regard to the environments in which different embodiments can be implemented. Many modifications to the depicted environment can be made. Computing device 400 includes processor(s) 404, memory 406, cache 416, communications fabric 402, persistent storage 408, input/output (I/O) interface(s) 412, and communications unit 410. Communications fabric 402 provides communications between memory 406, cache 416, persistent storage 408, input/output (I/O) interface(s) 412, and communications unit 410. Communications fabric 402 can be implemented with any architecture designed for passing data and/or control information between processors (such as microprocessors, communications and network processors, etc.), system memory, peripheral devices, and any other hardware components within a system. For example, communications fabric 402 can be implemented with one or more buses or a cross switch. Memory 406 and persistent storage 408 are computer readable storage media. In this embodiment, memory 406 includes random access memory (RAM). In general, memory 406 can include any suitable volatile or non-volatile computer readable storage media. Cache 416 is a fast memory that enhances the performance of computer processor(s) 404 by holding recently accessed data, and data near accessed data, from memory 406.

Program instructions and data (e.g., software and data) used to practice embodiments of the present invention may be stored in persistent storage 408 and in memory 406 for execution by one or more of the respective processor(s) 404 via cache 416. In an embodiment, persistent storage 408 includes a magnetic hard disk drive. Alternatively, or in addition to a magnetic hard disk drive, persistent storage 408 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 408 may also be removable. For example, a removable hard drive may be used for persistent storage 408. Other examples include optical and magnetic disks, thumb drives, and smart cards that are inserted into a drive for transfer onto another computer readable storage medium that is also part of persistent storage 408. Software and data can be stored in persistent storage 408 for access and/or execution by one or more of the respective processor(s) 404 via cache 416. With respect to user computing device 130, software and data includes user interface 132. With respect to server 120, software and data includes program 122.

Communications unit 410, in these examples, provides for communications with other data processing systems or devices. In these examples, communications unit 410 includes one or more network interface cards. Communications unit 410 may provide communications through the use of either or both physical and wireless communications links. Program instructions and data (e.g., software and data) used to practice embodiments of the present invention may be downloaded to persistent storage 408 through communications unit 410.

I/O interface(s) 412 allows for input and output of data with other devices that may be connected to each computer system. For example, I/O interface(s) 412 may provide a connection to external device(s) 418, such as a keyboard, a keypad, a touch screen, and/or some other suitable input device. External device(s) 418 can also include portable computer readable storage media, such as, for example, thumb drives, portable optical or magnetic disks, and memory cards. Program instructions and data (e.g., software and data) used to practice embodiments of the present invention can be stored on such portable computer readable storage media and can be loaded onto persistent storage 408 via I/O interface(s) 412. I/O interface(s) 412 also connect to display 420.

Display 420 provides a mechanism to display data to a user and may be, for example, a computer monitor.

The programs described herein are identified based upon the application for which they are implemented in a specific embodiment of the invention. However, it should be appreciated that any particular program nomenclature herein is used merely for convenience, and thus the invention should not be limited to use solely in any specific application identified and/or implied by such nomenclature.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

While particular embodiments of the present invention have been shown and described here, it will be understood to those skilled in the art that, based upon the teachings herein, changes and modifications may be made without departing from the embodiments and its broader aspects. Therefore, the appended claims are to encompass within their scope all such changes and modifications as are within the true spirit and scope of the embodiments. Furthermore, it is to be understood that the embodiments are solely defined by the appended claims. It will be understood by those with skill in the art that if a specific number of an introduced claim element is intended, such intent will be explicitly recited in the claim, and in the absence of such recitation no such limitation is present. For a non-limiting example, as an aid to understand, the following appended claims contain usage of the introductory phrases “at least one” and “one or more” to introduce claim elements. However, the use of such phrases should not be construed to imply that the introduction of a claim element by the indefinite articles “a” or “an” limits any particular claim containing such introduced claim element to embodiments containing only one such element, even when the same claim includes the introductory phrases “at least one” or “one or more” and indefinite articles such as “a” or “an”, the same holds true for the use in the claims of definite articles.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart illustrations and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart illustrations and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart illustrations and/or block diagram block or blocks.

The flowchart illustrations and block diagrams in the Figures illustrate the architecture, functionality, and operation of possible implementations of systems, methods, and computer program products according to various embodiments of the present invention. In this regard, each block in the flowchart illustrations or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each flowchart illustration and/or block of the block diagrams, and combinations of flowchart illustration and/or blocks in the block diagrams, can be implemented by special purpose hardware-based systems that perform the specified functions or acts or carry out combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer-implemented method comprising:
receiving, by one or more processors, a request to predict an effectiveness of a given social program for a given patient at one or more points in time from a user;
responsive to determining a social program database contains historical data on the given social program, analyzing, by the one or more processors, a set of patients associated with the given social program;
predicting, by the one or more processors, the effectiveness of the given social program for the given patient at the one or more points in time using a prediction model trained to predict an effectiveness score and a confidence score; and
outputting, by the one or more processors, a prediction of the effectiveness of the given social program for the given patient at the one or more points in time to the user.

2. The computer-implemented method of claim 1, further comprising:
requesting, by the one or more processors, feedback on the prediction of the effectiveness of the given social program for the given patient at the one or more points in time;
receiving, by the one or more processors, the feedback on the prediction of the effectiveness of the given social program for the given patient at the one or more points in time; and
integrating, by the one or more processors, the feedback on the prediction of the effectiveness of the given social program for the given patient at the one or more points in time to refine the prediction model.

3. The computer-implemented method of claim 1, wherein patient data includes socio-demographic information of the given patient, an Electronic Health Record of the given patient, a record of past and present social program the given patient participated in, and one or more Activities of Daily Living of the given patient.

4. The computer-implemented method of claim 1, wherein the historical data on the given social program includes an enrollment history of the given social program and one or more previous outcomes of the given social program.

5. The computer-implemented method of claim 1, wherein analyzing the set of patients associated with the given social program further comprises:
calculating, by the one or more processors, a first measure of similarity between a profile of the given patient and each profile of the set of patients associated with the given social program; and
selecting, by the one or more processors, a subset of patients from the set of patients associated with the given social program who exceed a pre-set threshold of similarity.

6. The computer-implemented method of claim 1, wherein the prediction of the effectiveness of the given social program for the given patient at the one or more points in time is comprised of the effectiveness score, the confidence score, and an explanation of the prediction of the effectiveness of the given social program for the given patient at the one or more points in time, wherein the explanation of the prediction of the effectiveness of the given social program for the given patient at the one or more points in time includes one or more factors used to calculate the effectiveness score.

7. The computer-implemented method of claim 1, wherein predicting the effectiveness of the given social program for the given patient at the one or more points in time using the prediction model trained to predict the effectiveness score and the confidence score further comprises:
defining, by the one or more processors, a first indicator function for each patient of the subset of patients selected at the one or more points in time using a default function;
aggregating, by the one or more processors, a plurality of first indicator functions defined for each patient of the subset of patients selected at the one or more points in time;
calculating, by the one or more processors, the effectiveness score using a value of the plurality of first indicator functions aggregated;
calculating, by the one or more processors, the confidence score using the value of the plurality of first indicator functions aggregated;
compiling, by the one or more processors, an explanation of one or more factors that contributed to a calculation of the effectiveness score and a calculation of the confidence score using domain knowledge; and
compiling, by the one or more processors, the explanation of the one or more factors that contributed to the calculation of the effectiveness score and the calculation of the confidence score using a machine learning explanation and interpretation technique.

8. The computer-implemented method of claim 7, wherein the default function is a historical outcome of the given social program for the subset of patients from the set of patients associated with the given social program.

9. The computer-implemented method of claim 1, further comprises:
responsive to determining the social program database does not contain historical data on the given social program, identifying, by the one or more processors, a set of social programs similar to the given social program;
calculating, by the one or more processors, a second measure of similarity between a feature of the given social program and a feature of each social program in the set of social programs similar to the given social program;
selecting, by the one or more processors, a subset of social programs from the set of social programs similar to the given social program that exceed the pre-set threshold of similarity;
analyzing, by the one or more processors, the set of patients associated with the subset of social programs selected;
calculating, by the one or more processors, a third measure of similarity between the profile of the given patient and each profile of the set of patients associated with the subset of social programs selected;
selecting, by the one or more processors, a subset of patients from the set of patients associated with the subset of social programs selected who exceed the pre-set threshold of similarity;
predicting, by the one or more processors, the effectiveness of the given social program for the given patient at the one or more points in time using the prediction model trained to predict the effectiveness score and the confidence score; and
outputting, by the one or more processors, the prediction of the effectiveness of the given social program for the given patient at the one or more points in time to the user.

10. The computer-implemented method of claim 9, wherein predicting the effectiveness of the given social program for the given patient at the one or more points in time using the prediction model trained to predict the effectiveness score and the confidence score further comprises:
defining, by the one or more processors, a second indicator function for the subset of patients selected and the subset of social programs selected at the one or more points in time using the default function;
aggregating, by the one or more processors, a plurality of second indicator functions defined for the subset of patients selected and the subset of social programs selected at the one or more points in time;
calculating, by the one or more processors, the effectiveness score using a value of the plurality of second indicator functions aggregated;
calculating, by the one or more processors, the confidence score using the value of the plurality of second indicator functions aggregated;
compiling, by the one or more processors, the explanation of the one or more factors that contributed to the calculation of the effectiveness score and the calculation of the confidence score at the one or more points in time using domain knowledge; and
compiling, by the one or more processors, the explanation of the one or more factors that contributed to the calculation of the effectiveness score and the calculation of the confidence score at the one or more points in time using the machine learning explanation and interpretation technique.

11. A computer program product comprising:
one or more computer readable storage media and program instructions stored on the one or more computer readable storage media, the program instructions comprising:
program instructions to receive a request to predict an effectiveness of a given social program for a given patient at one or more points in time from a user;
responsive to determining a social program database contains historical data on the given social program, program instructions to analyze a set of patients associated with the given social program;
program instructions to predict the effectiveness of the given social program for the given patient at the one or more points in time using a prediction model trained to predict an effectiveness score and a confidence score; and
program instructions to output a prediction of the effectiveness of the given social program for the given patient at the one or more points in time to the user.

12. The computer program product of claim 11, wherein analyzing the set of patients associated with the given social program further comprises:
program instructions to calculate a first measure of similarity between a profile of the given patient and each profile of the set of patients associated with the given social program; and
program instructions to select a subset of patients from the set of patients associated with the given social program who exceed a pre-set threshold of similarity.

13. The computer program product of claim 11, wherein predicting the effectiveness of the given social program for the given patient at the one or more points in time using the prediction model trained to predict the effectiveness score and the confidence score further comprises:
program instructions to define a first indicator function for each patient of the subset of patients selected at the one or more points in time using a default function;
program instructions to aggregate a plurality of first indicator functions defined for each patient of the subset of patients selected at the one or more points in time;
program instructions to calculate the effectiveness score using a value of the plurality of first indicator functions aggregated;
program instructions to calculate the confidence score using the value of the plurality of first indicator functions aggregated;
program instructions to compile an explanation of one or more factors that contributed to a calculation of the effectiveness score and a calculation of the confidence score using domain knowledge; and
program instructions to compile the explanation of the one or more factors that contributed to the calculation of the effectiveness score and the calculation of the confidence score using a machine learning explanation and interpretation technique.

14. The computer program product of claim 11, further comprises:
responsive to determining the social program database does not contain historical data on the given social program, program instructions to identify a set of social programs similar to the given social program;
program instructions to calculate a second measure of similarity between a feature of the given social program and a feature of each social program in the set of social programs similar to the given social program;
program instructions to select a subset of social programs from the set of social programs similar to the given social program that exceed the pre-set threshold of similarity;
program instructions to analyze the set of patients associated with the subset of social programs selected;
program instructions to calculate a third measure of similarity between the profile of the given patient and each profile of the set of patients associated with the subset of social programs selected;
program instructions to select a subset of patients from the set of patients associated with the subset of social programs selected who exceed the pre-set threshold of similarity;
program instructions to predict the effectiveness of the given social program for the given patient at the one or more points in time using the prediction model trained to predict the effectiveness score and the confidence score; and
program instructions to output the prediction of the effectiveness of the given social program for the given patient at the one or more points in time to the user.

15. The computer program product of claim 14, wherein predicting the effectiveness of the given social program for the given patient at the one or more points in time using the prediction model trained to predict the effectiveness score and the confidence score further comprises:
program instructions to define a second indicator function for the subset of patients selected and the subset of social programs selected at the one or more points in time using the default function;
program instructions to aggregate a plurality of second indicator functions defined for the subset of patients selected and the subset of social programs selected at the one or more points in time;
program instructions to calculate the effectiveness score using a value of the plurality of second indicator functions aggregated;
program instructions to calculate the confidence score using the value of the plurality of second indicator functions aggregated;
program instructions to compile the explanation of the one or more factors that contributed to the calculation of the effectiveness score and the calculation of the confidence score at the one or more points in time using domain knowledge; and
program instructions to compile the explanation of the one or more factors that contributed to the calculation of the effectiveness score and the calculation of the confidence score at the one or more points in time using the machine learning explanation and interpretation technique.

16. A computer system comprising:
one or more computer processors;
one or more computer readable storage media;
program instructions collectively stored on the one or more computer readable storage media for execution by at least one of the one or more computer processors, the stored program instructions comprising:
program instructions to receive a request to predict an effectiveness of a given social program for a given patient at one or more points in time from a user;
responsive to determining a social program database contains historical data on the given social program, program instructions to analyze a set of patients associated with the given social program;
program instructions to predict the effectiveness of the given social program for the given patient at the one or more points in time using a prediction model trained to predict an effectiveness score and a confidence score; and
program instructions to output a prediction of the effectiveness of the given social program for the given patient at the one or more points in time to the user.

17. The computer system of claim 16, wherein analyzing the set of patients associated with the given social program further comprises:
program instructions to calculate a first measure of similarity between a profile of the given patient and each profile of the set of patients associated with the given social program; and
program instructions to select a subset of patients from the set of patients associated with the given social program who exceed a pre-set threshold of similarity.

18. The computer system of claim 16, wherein predicting the effectiveness of the given social program for the given patient at the one or more points in time using the prediction model trained to predict the effectiveness score and the confidence score further comprises:
program instructions to define a first indicator function for each patient of the subset of patients selected at the one or more points in time using a default function;
program instructions to aggregate a plurality of first indicator functions defined for each patient of the subset of patients selected at the one or more points in time;
program instructions to calculate the effectiveness score using a value of the plurality of first indicator functions aggregated;
program instructions to calculate the confidence score using the value of the plurality of first indicator functions aggregated;
program instructions to compile an explanation of one or more factors that contributed to a calculation of the effectiveness score and a calculation of the confidence score using domain knowledge; and
program instructions to compile the explanation of the one or more factors that contributed to the calculation of the effectiveness score and the calculation of the confidence score using a machine learning explanation and interpretation technique.

19. The computer system of claim 16, further comprises:
responsive to determining the social program database does not contain historical data on the given social program, program instructions to identify a set of social programs similar to the given social program;
program instructions to calculate a second measure of similarity between a feature of the given social program and a feature of each social program in the set of social programs similar to the given social program;
program instructions to select a subset of social programs from the set of social programs similar to the given social program that exceed the pre-set threshold of similarity;
program instructions to analyze the set of patients associated with the subset of social programs selected;
program instructions to calculate a third measure of similarity between the profile of the given patient and each profile of the set of patients associated with the subset of social programs selected;
program instructions to select a subset of patients from the set of patients associated with the subset of social programs selected who exceed the pre-set threshold of similarity;
program instructions to predict the effectiveness of the given social program for the given patient at the one or more points in time using the prediction model trained to predict the effectiveness score and the confidence score; and
program instructions to output the prediction of the effectiveness of the given social program for the given patient at the one or more points in time to the user.

20. The computer system of claim 19, wherein predicting the effectiveness of the given social program for the given patient at the one or more points in time using the prediction model trained to predict the effectiveness score and the confidence score further comprises:
program instructions to define a second indicator function for the subset of patients selected and the subset of social programs selected at the one or more points in time using the default function;
program instructions to aggregate a plurality of second indicator functions defined for the subset of patients selected and the subset of social programs selected at the one or more points in time;
program instructions to calculate the effectiveness score using a value of the plurality of second indicator functions aggregated;
program instructions to calculate the confidence score using the value of the plurality of second indicator functions aggregated;
program instructions to compile the explanation of the one or more factors that contributed to the calculation of the effectiveness score and the calculation of the confidence score at the one or more points in time using domain knowledge; and
program instructions to compile the explanation of the one or more factors that contributed to the calculation of the effectiveness score and the calculation of the confidence score at the one or more points in time using the machine learning explanation and interpretation technique.
